(19) United States
(12) Patent Application Publication
(10) Pub. No.: US 2013/0343996 A1
(43) Pub. Date: Dec. 26, 2013

(54) GRAPHITE-COATED MAGNETIC NANOPARTICLES

(71) Applicant: Rutgers, The State University of New Jersey, (US)

(72) Inventors: Ki-Bum Lee, Monmouth Junction, NJ (US); Prasad Subramaniam, Edison, NJ (US)

(73) Assignee: Rutgers, The State University of New Jersey, New Brunswick, NJ (US)

(21) Appl. No.: 13/870,625

(22) Filed: Apr. 25, 2013

Related U.S. Application Data
(60) Provisional application No. 61/637,912, filed on Apr. 25, 2012.

(51) Int. Cl. A61K 9/14; A61K 41/00; A61K 38/02; A61K 49/00; A61K 31/713; A61K 39/395

(52) U.S. Cl. CPC A61K 9/143; A61K 31/713; A61K 39/39558; A61K 38/02; A61K 49/00; A61K 41/0052
USPC 424/9.1; 514/44 A; 424/493; 424/174.1; 514/19.3; 424/143.1

(57) ABSTRACT

The present invention relates to graphite-coated magnetic nanoparticles, methods for the synthesis of graphite-coated magnetic nanoparticles, and methods of using graphite-coated magnetic nanoparticles for targeted delivery of siRNA-based therapy, as multimodal imaging probes and for hyperthermia cancer treatment.

### CROSS REFERENCE TO RELATED APPLICATION

This application claims the benefit of U.S. Provisional Application No. 61/637,912 filed Apr. 25, 2012, the disclosure of which is incorporated herein by reference in its entirety.

### STATEMENT REGARDING FEDERALLY SPONSORED RESEARCH

The invention described herein was supported in whole or in part by grants from the National Institutes of Health (NIH Director's Innovator Award No. NIH-1DP20D006462-01). The U.S. Government has certain rights in this invention.

### FIELD OF THE INVENTION

The present invention relates to graphite-coated magnetic nanoparticles, the synthesis of graphite-coated magnetic nanoparticles, and methods of using graphite-coated magnetic nanoparticles for targeted delivery of siRNA-based therapy, as multimodal imaging probes and for hyperthermia cancer treatment.

### BACKGROUND OF THE INVENTION

Cancer treatment generally requires the combination of several modalities such as chemotherapy, radiation, and hyperthermia. The development of multifunctional nanomaterial-based systems with combined therapeutic and molecular imaging capabilities are mostly focused on the synthesis and characterization of materials with limited demonstration of their use for biomedical applications. As a result, research efforts towards developing magnetic nanoparticle based multimodal therapeutics to control the tumor microenvironment are highly limited. Therefore, to address the challenges of magnetic nanoparticle based therapeutics as well as to narrow the gap between current nanoparticle-based multimodal imaging approaches and their clinical applications, there is a clear need to synthesize effective chemotherapeutic magnetic nanoparticles and to develop multimodal therapies for targeting specific oncogenes, thereby activating/deactivating corresponding key signaling pathways.

These and other aspects of the present invention will be better appreciated by reference to the following drawings and detailed description.

### SUMMARY OF THE INVENTION

The present invention relates to graphite-coated magnetic nanoparticles, methods for the synthesis of graphite-coated magnetic nanoparticles, and methods of using graphite-coated magnetic nanoparticles for targeted delivery of siRNA-based therapy, as multimodal imaging probes and for hyperthermia cancer treatment. This invention fulfills the need for effective chemotherapeutic magnetic nanoparticles for targeting specific oncogenes, thereby activating/deactivating corresponding key signaling pathways.

In one aspect, the invention provides a nanoparticle comprising an iron cobalt core; a graphitic carbon shell surrounding the core; a biocompatible carbohydrate coating disposed on the shell; a cationic coating disposed on said biocompatible coating; and small interfering RNAs (siRNAs) disposed on said cationic coating, wherein said siRNAs are selected to interfere with expression of a gene in a tumor cell. The biocompatible coating may comprise functional groups for covalent attachment of tumor targeting agents; and said nanoparticle may further comprise a targeting agent for said tumor cell conjugated to one of said functional groups. The carbohydrate may be dextran. The targeting agent may be an antibody, a peptide, a carbohydrate, a lipopolysaccharide, or a small molecule. The antibody may be a monoclonal antibody that binds to an epidermal growth factor receptor. The peptide may contain a RGD sequence. The cationic coating may be polyethyleneimine.

In a second aspect, the invention provides a pharmaceutical composition comprising the nanoparticle as previously described above and a pharmaceutically acceptable carrier.

In a third aspect, the invention provides a method to deliver a siRNA to the intracellular region of a cell of interest in a subject comprising administering a pharmaceutical composition comprising the nanoparticle as previously described. The targeting agent may target an epidermal growth factor receptor or a receptor that specifically binds to a peptide containing RGD. The cell may be a glioblastoma cell.

In a fourth aspect, the invention provides a method to increase the temperature of a cell in a subject comprising administering a pharmaceutical composition comprising the nanoparticle as previously described above and applying a magnetic field to the cells targeted by the targeting agent. A magnetic field may be applied using a radiofrequency field generator.

In a fifth aspect, the invention provides a method to silence at least one gene in a subject comprising administering a pharmaceutical composition comprising a nanoparticle as previously described above and a pharmaceutically acceptable carrier. The gene may be an epidermal growth factor receptor gene.

In a sixth aspect, the invention provides a method to detect a cell in a subject comprising administering a pharmaceutical composition comprising a nanoparticle as previously described above and detecting said nanoparticle using an imaging device. The imaging device may be a magnetic resonance imaging device or a Raman spectroscopy device.

In a seventh aspect, the invention provides a kit for detecting a cell in a biological sample or a subject comprising a nanoparticle as previously described above and instructions for use.

In an eighth aspect, the invention provides a method of preparing a nanoparticle comprising an iron cobalt core and a graphitic carbon shell surrounding the core, the method comprising the steps of: dissolving iron and cobalt precursors with a carbohydrate in an aqueous solution; heating the solution under conditions sufficient for formation of nanoparticles; and annealing the nanoparticles under conditions suitable for the formation of a graphitic carbon shell on the nanoparticles. The method of preparing a nanoparticle may further comprise suspending the nanoparticles with a carbohydrate under basic conditions to provide a biocompatible coating on said shell; magnetically separating the coated nanoparticles from the solution; adding an aqueous solution of a cationic polymer to the biocompatible coated nanoparticles under conditions to provide a cationic coating on the biocompatible coated nanoparticles; purifying the nanoparticles; adding a solution of an siRNA to the nanoparticles. Optionally the method may comprise repeating the following steps to provide additional layers of a cationic coating, adding an aqueous solution of a cationic polymer to the biocompatible coated nanoparticles under conditions to provide a cationic coating on the biocompatible coated nanoparticles; and purifying the nanoparticles. The aqueous solution may be water. The nanoparticles may be exposed heating conditions at a temperature between about 180-250° F. for about 7-10 hours or to an argon atmosphere at a temperature between about 900-1100° F. for about 4-6 hours.

### BRIEF DESCRIPTION OF THE DRAWINGS

FIGS. 1a and b depict magnetic FeCo-graphite nanoparticles for multimodal imaging and targeted tumor therapy. a) Detailed structure of the MNPs depicting the highly magnetic FeCo core, protective Raman active graphite shell and the biocompatible dextran coating b) Inhibition of proliferation and induction of apoptosis via combined siRNA delivery and hyperthermia using siRNA-FeCo/C NP constructs.

FIGS. 2a-d provide a schematic diagram depicting the conjugation strategies for attaching biomolecules to the surface of FeCo/C nanoparticles. a) Synthesis of dextran derivatives terminated with 4-nitrophenyl chloroformate group (R1), —NH₂ group (R2) and —COOH group (R3). b) Coating of the FeCo/C NPs with dextran derivatives to render them biocompatible and enable conjugation of biomolecules onto their surface. c) and d) Conjugation of EGFR antibody and cRGD peptide to the reactive functional groups present on the dextran coated FeCo/C NPs.

### DETAILED DESCRIPTION OF THE INVENTION

The present invention relates to graphite-coated magnetic nanoparticles, methods for the synthesis of graphite-coated magnetic nanoparticles, and methods of using graphite-coated magnetic nanoparticles for targeted delivery of siRNA-based therapy, as multimodal imaging probes and for hyperthermia cancer treatment.

Nanoparticles

In one embodiment, the present invention provides a nanoparticle comprising an iron cobalt (FeCo) core; a graphitic carbon shell surrounding the core; a biocompatible carbohydrate coating disposed on the shell; a cationic coating disposed on said biocompatible coating; and small interfering RNAs (siRNAs) disposed on said cationic coating, wherein said siRNAs are selected to interfere with expression of a gene in a tumor cell.

A nanoparticle comprising an iron cobalt core with a graphitic carbon shell surrounding the core (FeCo/C) may be synthesized by hydrothermal synthetic methods, followed by an annealing process at high temperatures. The NPs are magnetic and can thus be detected by imaging devices, and can also be heated by devices that emit a magnetic field. The present invention provides synthetic methods that are cost effective, easy to scale up, exclude toxic solvents, and as a result provide for milder synthetic conditions with a low environmental impact. Also, the synthetic methods allow for greater yields of NPs and the ability to vary the thickness of the graphitic shell and/or number of graphitic shells surrounding the iron cobalt core. The thickness of the graphitic shell and/or number of graphitic shells surrounding the iron cobalt core can be adjusted by varying the amount of a carbohydrate, such as sucrose. A thicker graphitic shell can facilitate an improved Raman signal intensity for detecting cancer cells. For illustration purposes, FeCo carbon (FeCo/C) nanoparticles prepared by dissolving 6 mmol of a Fe precursor and 4 mmol of a Co precursor with 2.9 mmol of sucrose yielded a NP with a diameter of 7 nm, whereas a FeCo/C nanoparticle with the same precursor concentrations prepared using 8.8 mmol of sucrose yielded a NP with a diameter of 11 nm, a graphite shell that is twice as thick. One with ordinary skill in the art can vary and adjust the desired thickness of the surrounding graphitic shell.

The FeCo/C NPs may be prepared by dissolving iron and cobalt precursors in an aqueous solution, adding a carbohydrate to the solution, heating the solution under conditions suitable for formation of nanoparticles, and annealing the nanoparticles under conditions suitable for the formation of a graphite shell on the nanoparticle. In a preferred embodiment, the aqueous solution is water and the solution of precursors is heated at a temperature between 180-250° F. for about 7-10 hours to form nanoparticles. In another preferred embodiment, heating the solution comprises drying in an oven at a temperature between 60-100° F. for about 4-6 hours. In another preferred embodiment, the nanoparticles are washed and dried prior to the annealing step. In another preferred embodiment, the nanoparticles are annealed under an argon atmosphere at a temperature between 900-1100° F. for 4-6 hours to form the graphite shell of the nanoparticle. Large amounts of FeCo/C NPs may be prepared by the steps of dissolving iron and cobalt precursors in an aqueous solution, adding a carbohydrate to the solution, and heating the solution under conditions suitable for formation of nanoparticles by one with ordinary skill in the art to obtain a yield of equal to or greater than 95%.

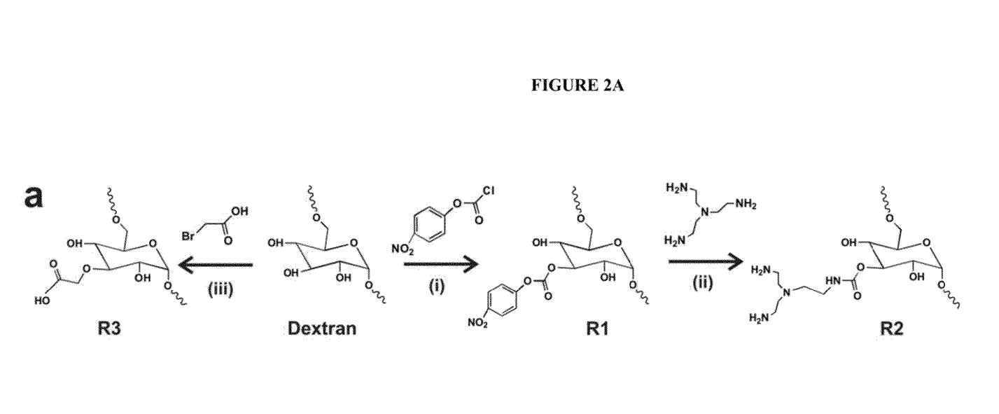

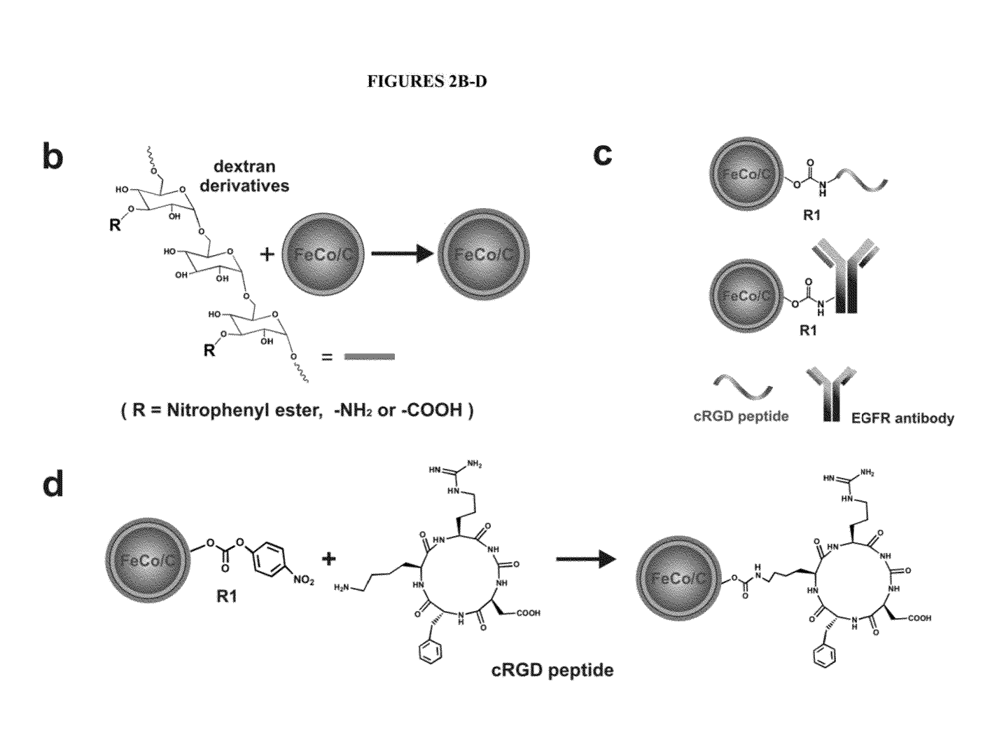

The FeCo/C NP further comprises a biocompatible carbohydrate coating disposed on the graphitic shell. In a preferred embodiment the carbohydrate is dextran, however other carbohydrates that have similar water solubility, biocompatibility, and biodegradability properties may be used as known in the art. The biocompatible carbohydrate coating may comprise various functionized carbohydrates. In a preferred embodiment, a functionalized carbohydrate may contain a 4-nitrophenyl chloroformate groups, an amine group, or a carboxyl group. In a preferred embodiment, the biocompatible carbohydrate coating comprises dextran functionalized with 4-nitrophenyl chloroformate groups (4-NC dextran), amine groups, and/or carboxyl groups (FIG. 2). The carbohydrates may be disposed on the graphitic shell by suspending the FeCo/C NPs and the carbohydrates under basic conditions such as NaOH, to provide a biocompatible coating on the shell. Thereafter the FeCo/C NPs may be exposed to sonification, magnetically separated from the solution, then washed with water to remove unreacted carbohydrates. The average hydrodynamic diameter of a FeCo/C NP with a biocompatible coating is about 120-160 nm.

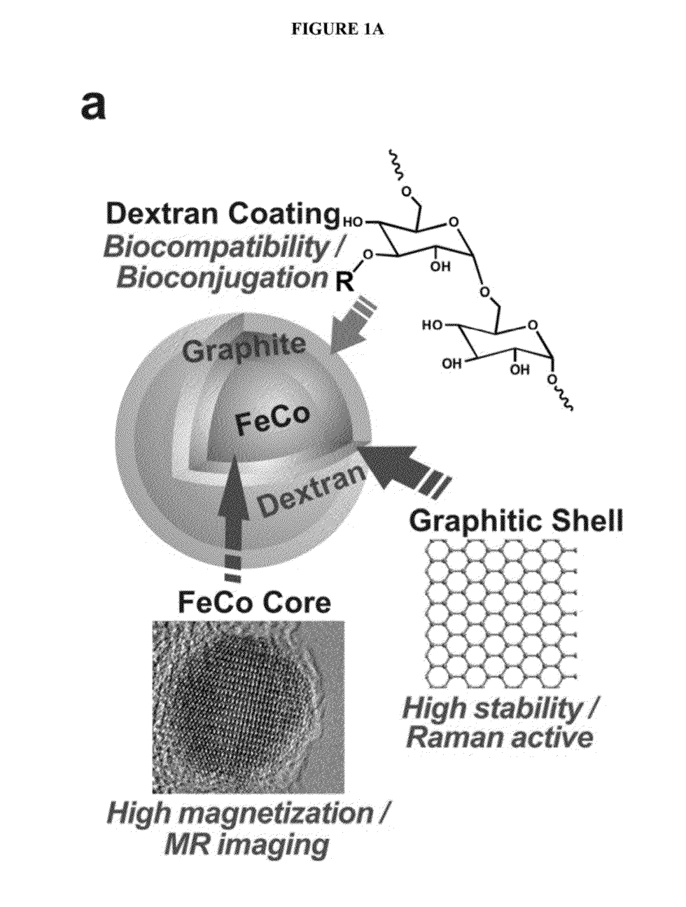

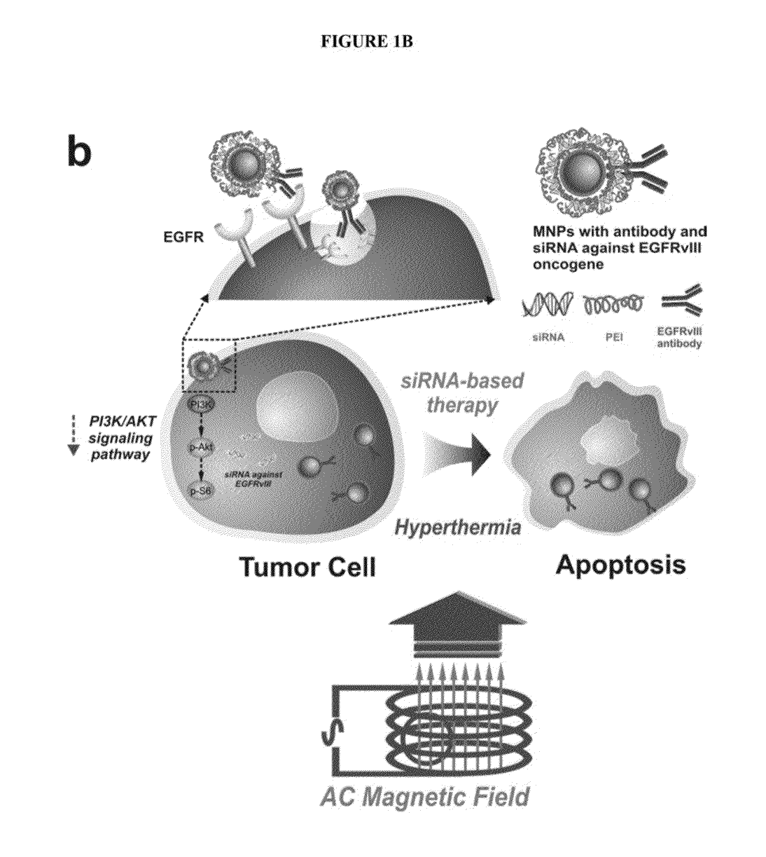

The FeCo/C NP further comprises a cationic coating. In a preferred embodiment, the cationic coating comprises a cationic polymer, such as polyethylenimine (PEI), as a ligand coating to capture a small interfering RNA (siRNA). Other polymers, carbohydrates and peptides, and combinations thereof that may be used as a cationic coating include DeXAM (cyclodextrin-conjugated dendrimer), Poly-amidoamine (PAMAM, dendrimer), Poly-L-Lysine (PLL, A polypeptide of L-Lysine), Poly-L-Arginine (A polypeptide of L-arginine), Chitosan (a glycopolysaccharide), Poly(beta-amino esters), Poly(4-hydroxy-1-proline ester) (PHP), Poly(2-dimethylaminoethyl methacrylate) (pDMAEMA). For additional materials that may be used for the cationic coating, see Luten et all, J. of Controlled Release 126 (2008) 97-110. FeCo/C NPs are added to an aqueous solution of a cationic polymer to the biocompatible coated nanoparticles under conditions to provide a cationic coating on the biocompatible coated nanoparticles, then purified. Then a solution of siRNAs is added to a solution of FeCo/C NPs with the cationic coating, and then optionally, the FeCo/C NPs are purified, and another layer of a cationic coating is deposited on the siRNA layer of the FeCo/C NPs (see FIG. 1b).

Small interfering RNAs are small double stranded RNAs (dsRNAs) that optionally include overhangs. For example, the duplex region of an siRNA may be from about 18 to 25 nucleotides in length, e.g., about 19, 20, 21, 22, 23, or 24 nucleotides in length. Typically, the siRNA sequences are exactly complementary to the target mRNA, although some mismatch may be tolerated. The dsRNAs and siRNAs in particular can be used to silence gene expression in mammalian cells (e.g. human cells). See, e.g., Clemens et al. (2000) Proc. Natl. Acad. Sci. USA 97:6499-6503: Billy et al. (2001) Proc. Natl. Sci. USA 98:14428-44433; Elbashir et al. (2001) Nature 411:494-498; Yang et al. (2002) Proc. Natl. Acad. Sci. USA 99:99429947, U.S. Pub App. Nos. 2003-0166282; 2003-0143204: 2004-0038278; and 2003-0224432.

In a preferred embodiment, a first layer of the cationic coating surrounds the biocompatible carbohydrate coating, and a layer of nucleic acids is disposed on the first layer of cationic coating, with a second layer of the cationic coating surrounding the nucleic acids and the FeCo/C NP (See FIG. 1). In a preferred embodiment the cationic coating is a polymer with similar properties to polyethyleneimine. In a preferred embodiment, the nucleic acid is at least one siRNA that silences a gene that is upregulated in a cancer cell, e.g. the epidermal growth factor receptor gene (EGFR) or mutated EGFR gene associated with cancer such as EGFRvIII. In a further embodiment various different siRNAs may be selected in which the expression of more than one gene may be silenced. One with ordinary skill in the art can select a gene that is upregulated in a cancer cell to be silenced, and also select a siRNA to silence the gene. In certain embodiments, at least one targeting agent is also covalently attached to the biocompatible carbohydrate coating, and surrounded by the cationic coating, and capable of binding to the target receptor on the surface of a cancer cell.

The FeCo/C NP may further comprise a targeting agent. In a preferred embodiment, a targeting agent may be covalently attached to reactive functional groups of a functionalized carbohydrate within the biocompatible carbohydrate coating. The targeting agent may be covalently attached to the functionalized carbohydrate or attached by use of a linker. As used herein, the term “linker” refers to a chemical moiety that connects a molecule to another molecule, covalently links separate parts of a molecule or separate molecules. The linker provides spacing between the two molecules or moieties such that they are able to function in their intended manner. Examples of linking groups include peptide linkers, enzyme sensitive peptide linkers, self-immolative linkers, acid sensitive linkers, multifunctional organic linking agents, bifunctional inorganic crosslinking agents, polymers and other linkers known in the art. The linker may be stable or degradable/cleavable. Various linkers are known in the art.

The targeting agent may be an antibody, a peptide, a carbohydrate, a lipopolysaccharide, or a small molecule that targets a cancer marker such as a receptor or antigen expressed on the surface of a cancer cell, which may also improve intracellular uptake of the NP by the cell. The term “cancer” refers to or describes the physiological condition in mammals in which a population of cells are characterized by unregulated cell growth, and the population of cells are referred to as “cancer cells”. The term “tumor” as used herein refers to any mass of tissue that results from excessive cell growth or proliferation, either benign (noncancerous) or malignant (cancerous), including pre-cancerous lesions. Examples of cancer include, but are not limited to, epithelial cell cancer, breast cancer, prostate cancer, ovarian cancer, lung cancer, brain cancer, blood cancer, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, liver cancer, bladder cancer, hepatoma, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers. The terms “cancer cell,” and “tumor cell,” are used interchangeably and grammatical equivalents refer to the population of cells derived from a cancer, tumor or a pre-cancerous lesion. Examples of known cancer antigens/receptors expressed on the surface of a cancer cell that may be targeted by an antibody include the epidermal growth factor family (EGF-family), such as EGFR (ErbB-1), HER2/c-neu (ErbB-2), Her 3 (ErbB-3) and Her 4 (ErbB-4). Other targeted receptors include CD20, CD52, CD30, and VEGFa. In a preferred embodiment the targeted cell is a cancer nerve cell, e.g. a glioblastoma cell.

In a preferred embodiment, the antibody is a monoclonal antibody that targets a receptor expressed on the surface of a cancer cell. Examples of antibodies that target receptors expressed on the surface of a cancer cell or on the surface of a tumor cell include, alemtuzumab, bevacizumab, brentuximab, cetuximab, denosumab, gemtuzumab, ibritumomab, ipilimumab, ofatumumab, panitumumab, rituximab, and trastuzumab. In a preferred embodiment, the antibody binds to EGFR expressed on cancer cells. The term “antibody” refers to an immunoglobulin or antigen-binding fragment thereof, and encompasses any such polypeptide comprising an antigen-binding fragment of an antibody. The term includes but is not limited to polyclonal, monoclonal, monospecific, polyspecific, humanized, human, single-chain, single-domain, chimeric, synthetic, recombinant, hybrid, mutated, grafted, and in vitro generated antibodies. The term “antibody” also includes antigen-binding fragments of an antibody. Examples of antigen-binding fragments include, but are not limited to, Fab fragments (consisting of the VL, VH, CL and CH1 domains); Fd fragments (consisting of the VH and CH1 domains); Fv fragments (referring to a dimer of one heavy and one light chain variable domain in tight, non-covalent association); dAb fragments (consisting of a VH domain); single domain fragments (VH domain, VL domain, VHH domain, or VNAR domain); isolated CDR regions; (Fab′)2 fragments, bivalent fragments (comprising two Fab fragments linked by a disulphide bridge at the hinge region), scFv (referring to a fusion of the VL and VII domains, linked together with a short linker), and other antibody fragments that retain antigen-binding function.

In certain embodiments, the targeting agent may be a carbohydrate that targets a cell surface receptor expressed on the surface of a cancer cell. The mannose receptor, which has a high affinity to D-mannose, is expressed on tumor-associated macrophages that enhance tumor progression by promoting tumor cell invasion, migration and angiogenesis. Other cancer cell surface receptors that bind to a carbohydrate are known in the art, as well as the carbohydrate ligand.

In certain embodiments the targeting agent may be a peptide that binds to a receptor expressed on the surface of a cancer cell. The terms “polypeptide”, “peptide”, “protein”, and “protein fragment” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. Peptides are known in the art to target receptors expressed on the surface of a cancer cell. Examples of such peptides include peptides that contain the RGD sequence, and/or cyclic RGD.

In certain embodiments the targeting agent may be a small molecule that binds to a receptor on the surface of a cancer cell. One such receptor known in the art is the folate receptor, and such small molecules that may be used as a targeting agent includes folate and various folate analogs. Other receptors expressed on the surface of a cancer cell, and small molecules that bind to such receptors are known in the art.

In a further embodiment, the present invention provides methods of using the FeCo/C NPs as hyperthermia agents to increase the temperature of a cell. Methods include contacting a cell with the NPs whereby the NPs are absorbed by the cell, e.g. by receptor mediated cytosis, and the NPs are exposed to an instrument that emits a magnetic field for a period of time. In a preferred embodiment the instrument is a radiofrequency field generator. One with ordinary skill in the art can determine the amount of time to expose the cell containing the biocompatible NPs to reach a desired temperature within the cell.

In a further embodiment, the present invention provides a method of preparing a nanoparticle as previously described comprising dissolving iron and cobalt precursors in an aqueous solution; adding a carbohydrate to the solution; heating the solution under conditions sufficient for formation of nanoparticles: washing the nanoparticles; drying the nanoparticles; annealing the nanoparticles under conditions sufficient to form a graphite shell on said nanoparticle; suspending the nanoparticles with a carbohydrate under basic conditions to provide a biocompatible coating on said shell; magnetically separating the coated nanoparticles from the solution: adding an aqueous solution of a cationic polymer to the biocompatible coated nanoparticles under conditions to provide a cationic coating on the biocompatible coated nanoparticles; purifying the nanoparticles; adding a solution of an siRNA to the nanoparticles; magnetically separating the coated nanoparticles from the solution; and optionally adding an aqueous solution of a cationic polymer to the biocompatible coated nanoparticles under conditions to provide additional layers of a cationic coating on the biocompatible coated nanoparticles; and purifying the nanoparticles.

### NP Pharmaceutical Composition

The NP compositions of the invention may be formulated as a pharmaceutical composition, and may be administered to a mammalian host, such as a human patient, in a variety of forms adapted to the chosen route of administration, i.e., orally or parenterally, by intravenous, intramuscular, topical, subcutaneous, or other routes. Thus, the pharmaceutical composition of the invention may be systemically administered, e.g., orally, in combination with a pharmaceutically acceptable vehicle such as an inert diluent. They may be incorporated directly with the food of the patient's diet. For oral therapeutic administration, the compositions of the invention may be used in the form of elixirs, syrups, and the like.

Any material used in preparing any unit dosage form should be pharmaceutically acceptable and substantially non-toxic in the amounts employed. To administer the pharmaceutical composition to a patient, it is preferable to formulate the molecules in a composition comprising one or more pharmaceutically acceptable carriers. The phrase “pharmaceutically acceptable” refers to molecular entities and compositions that do not produce allergic, or other adverse reactions when administered using routes well-known in the art. “Pharmaceutically acceptable carriers” include any and all clinically useful solvents, dispersion media, coatings, antibacterial and antifungal agents, isotonic and absorption delaying agents and the like.

The pharmaceutical composition of the present invention can be administered to a subject by any of a number of means known in the art. A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model. Typically, the terms “subject” and “patient” are used interchangeably herein in reference to a human subject.

The pharmaceutical composition of the invention may also be administered intravenously or intra-peritoneally by infusion or injection, among many other routes. Solutions may be prepared, for example, in water. However, other solvents may also be employed. Under ordinary conditions of storage and use, these preparations may contain a preservative to prevent the growth of microorganisms, and other formulation ingredients as is known in the art.

The pharmaceutical dosage forms suitable for injection or infusion should be preferably sterile, fluid and stable under the conditions of manufacture and storage. The prevention of the action of microorganisms may be brought about by various antibacterial and antifungal agents, for example, parabens, chlorobutanol, phenol, sorbic acid, thimerosal, and the like. Others are also suitable. In many cases, it may be preferable to include isotonic agents, for example, sugars, buffers or sodium chloride.

Sterile injectable solutions may be prepared by incorporating the pharmaceutical composition of the invention in the required amount into an appropriate solvent or medium with various other ingredients, e.g., those enumerated above, as needed, which may be followed by sterilization. The above-described pharmaceutical composition containing the nanoparticles can be used to treat cancer.

In a further embodiment, the present invention provides a method to deliver a siRNA to the intracellular region of a cell of interest comprising contacting a cell with a pharmaceutical composition comprising a nanoparticle comprising an iron cobalt core; a graphitic carbon shell surrounding the core; a functionalized biocompatible carbohydrate coating disposed on the shell; a cationic coating disposed on the biocompatible coating; and small interfering RNAs (siRNAs) disposed on said cationic coating, wherein said siRNAs are selected to interfere with expression of a gene in a tumor cell wherein the nanoparticle further comprises a targeting agent for said tumor cell conjugated to one of said functional groups.

In a preferred embodiment, the targeting agent may be a EGFR antibody or a peptide containing the RGD peptide and/or cyclic peptide, that targets the EGFR receptor of a gliablastoma cell. Furthermore, the gene may be a EGFR gene. One with ordinary skill in the art will select the appropriate siRNA to silence the EGFR gene of interest.

In a further embodiment, the present invention provides a method to detect a cell comprising contacting a cell in a biological sample with a pharmaceutical composition comprising a nanoparticle comprising an iron cobalt core; a graphitic carbon shell surrounding the core; a functionalized biocompatible carbohydrate coating disposed on the shell; a cationic coating disposed on the biocompatible coating; and small interfering RNAs (siRNAs) disposed on said cationic coating, wherein said siRNAs are selected to interfere with expression of a gene in a tumor cell wherein the nanoparticle further comprises a targeting agent for said tumor cell conjugated to one of said functional groups, and detecting the nanoparticle. The biological sample may contain a homogenous or heterogeneous collection of cells. One with ordinary skill in the art will adapt the proper modality to detect pharmaceutical compositions as described. Such modalities include magnetic resonance imaging and/or Raman spectroscopy.

In a further embodiment, the present invention provides a method to increase the temperature of a cell comprising contacting a cell in a biological sample with a pharmaceutical composition comprising a nanoparticle comprising an iron cobalt core; a graphitic carbon shell surrounding the core; a functionalized biocompatible carbohydrate coating disposed on the shell; a cationic coating disposed on the biocompatible coating; and small interfering RNAs (siRNAs) disposed on said cationic coating, wherein said siRNAs are selected to interfere with expression of a gene in a tumor cell wherein the nanoparticle further comprises a targeting agent for said tumor cell conjugated to one of said functional groups, and applying a magnetic field to the nanoparticle. The magnetic field may be applied using an instrument such as a radiofrequency field generator. The biological sample may contain a homogenous or heterogeneous collection of cells.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference in their entireties.

The following non-limiting examples set forth herein below illustrate certain aspects of the invention.

### EXAMPLES

### Synthesis of FeCo/C NPs

FeCo/C NPs (7 nm) were prepared by dissolving Fe(NO₃)₃.9H₂O (2.424 g, 6 mmol) and Co(NO₃)₂.6H₂O (1.164 g, 4 mmol) in 30 mL of distilled water and then 2.9 mmol of sucrose was added. The mixture was stirred vigorously to form a clear solution and then placed in a 45 mL capacity Teflon-lined stainless steel autoclave, which was heated in an oven to 190° C. for 9 h. For 11 nm FeCo/C NPs, the mixture was heated to 220° C. for 9 h. The products were washed several times with distilled water, filtered off and finally dried in a drying oven at 80° C. for 5 h. Subsequently, the dried products were annealed at 1000° C. for 3 h under Ar atmosphere to allow for the growth of the carbon graphite shell on the surface of FeCo NPs. To remove the remaining carbon graphite, the products were washed several times with distilled water and separated using a bar magnet. The number of graphite shells was controlled by varying the amount of sucrose between 2.9 mmol and 8.8 mmol. To prepare large amounts of FeCo/C magnetic NPs in one step, 13.5744 g of Fe(NO₃)₃.9H₂O (33.6 mmol) and 6.5184 g of Co(NO₃)₂.6H₂O (22.4 mmol) were added to 30 mL of distilled water, which was followed by the addition of 14.5 mmol of sucrose under the above mentioned reaction conditions. The amount of the FeCo/C NPs was as much as 5 g with a yield greater than 95%.

### Physical Characterization of the FeCo/C NPs:

The NPs were characterized using XRD (Bruker D8 X-ray diffractometer with CuKα radiation (Ni filter)), TEM (Tecnai G2 and JEOL JEM-2010F high-resolution transmission electron microscope operated at an accelerating voltage of 200 kV), Raman spectroscopy (Renishaw Micro-Raman 2000 with He—Ne laser excitation of 632.8 nm) and SQUID magnetometry (Quantum design magnetometer). The stoichiometry of Fe and Co and metal concentration in the NPs were determined using Inductively Coupled Plasma-Atomic Emission Spectrometer (ICP-AES, data not shown).

### Synthetic Routes to Functionalized Dextrans:

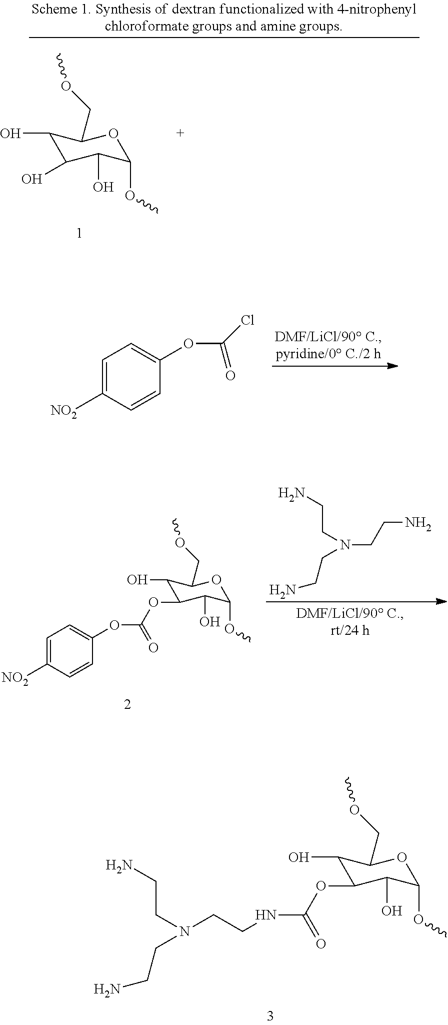

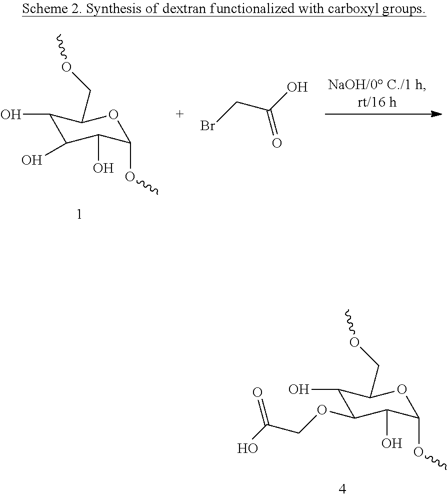

### Synthesis of Functionalized Dextrans:

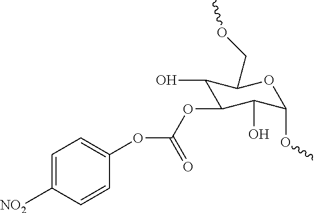

Dextran functionalized with 4-nitrophenyl Chloroformate Groups (4-NC Dextran) (2)

Scheme 1 shows the synthesis of 4-NC dextran (J. C. Ramirez, M. Sanchezchaves, F. Arranz, Angewandte Makromolekulare Chemie 1995, 225, 123-130). For a complete dissolution of dextran (1) in DMF, 15 g (92 mmol) dextran and 5.0 g (118 mmol, 2 w/v %) water-free lithium chloride (LiCl) were suspended in 250 ml DMF and stirred at 90° C. until a clear solution appears. The reaction mixture was cooled to 0° C., and pyridine (4.2 mL, 52 mmol) was added to the dextran solution followed by 4-NC (10.5 g, 52 mmol). The reaction mixture was stirred for 4 h at 0° C., and finally the 4-nitrophenyl carbonated dextran was isolated by precipitation in cold ethanol, washed with diethyl ether, and dried under vacuum. Yield: 14.5 g, 97%. ¹H NMR (D₂O): δ 3.27-4.13 (m, dextran glucosidic protons), 4.68 (s, dextran anomeric proton), 7.83 and 8.67 (dd, aromatic protons). (FIG. 20).

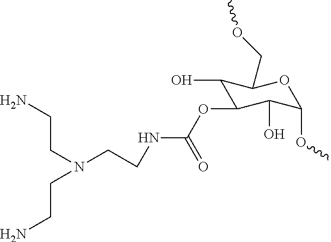

Dextran Functionalized with Amine Groups (3)

For a complete dissolution of 4-NC dextran in DMF, 7 g (37 mmol) 4-NC dextran and 1.13 g (26.7 mmol, 1 w/v %) water-free LiCl were suspended in 56 mL DMF and stirred at 90° C. until a clear solution appeared. The solution was then cooled again to 0° C. Tris(aminoethyl)amine (7 mL, 45 mmol) was dissolved in DMF (56 mL) and added drop wise under stirring to the 4-NC dextran solution. The reaction was stirred for 24 h at room temperature. Subsequently, the product was isolated by precipitation in cold ethanol and washed several times with ethanol to remove p-nitrophenol. The product was filtrated and dried under vacuum. Finally the completion of the reaction was determined by a Ninhydrin color test and NMR spectroscopy. Yield: 4.6 g, 66%. ¹H NMR (DMSO-d6): δ 1.0 (t, amine protons), δ 2.42-2.57 (m, amine protons), δ 3.1-3.8 (m, dextran glucosidic protons), 4.08 (s, dextran glucosidic protons), 4.69 (s, dextran anomeric proton), 8.0 (s, amide protons). (FIG. 21).

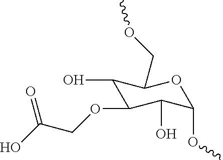

Dextran Functionalized with Carboxyl Groups (4)

Scheme 2 shows the representative synthesis for obtaining dextran functionalized by carboxyl groups (4). Carboxydextran was synthesized using the methods reported previously (S. Lee, V. H. Perez-Luna, Analytical Chemistry 2005, 77, 7204-7211). 6 g dextran was dissolved in 20 mL 0.1 M NaOH. Then IM bromoacetic acid (1.4 g) and 10 mL 2 M NaOH (0.8 g) were added to 10 mL of the dextran solution under stirring. The solution was stirred for 24 h at room temperature. The carboxydextran was then isolated by precipitation in acetone and subsequently washed with acetone. The product was dried under vacuum. Yield: 5.8 g, 96%. ¹H NMR (D₂O): δ 3.26-4.18 (m, dextran glucosidic protons), 4.52-4.92 (m, dextran anomeric proton), 5.05-5.10 (s, carboxylic acid protons). (FIG. 22).

Surface Modification of the FeCo/C NPs with the Functionalized Dextrans

200 mg of FeCo/C magnetic NPs were dispersed in 10 ml of NaOH (0.5 M) and this suspension was mixed with a solution of 400 mg of functionalized dextran in NaOH (0.5 M) prepared previously. The mixture was sonicated for 24 h at 30° C. and the coated NPs were separated magnetically and washed thrice with water to remove the unreacted functionalized dextran.

### T₂ Measurements

Aqueous solutions of varying concentrations of FeCo/C NPs were used for T₂ measurements using a 4.7 T MRI instrument with a 72 mm volume coil (Bruker, Germany). The T₂ values of various phantom solutions were obtained from the Carr-Purcell-Meiboom-Gill (CPMG) sequence at room temperature (TR=10 s, 128 echoes with 7.4 ms even echo space, number of acquisition=1, spatial resolution=391 μm×391 μm, section thickness=1 mm).

### Conjugation of Targeting Molecules to FeCo/C NPs

Amine reactive FeCo/C-Np-Dex (Nitrophenyl ester conjugated dextran; Np-Dex) magnetic NPs were dispersed in degassed PBS. To 50 μl of the above FeCo/C solution (9 mg/ml), 20 μl of EGFR (Epidermal Growth Factor Receptor) antibody (0.5 mg/ml, BD Pharmingen, San Diego, Calif.) or 25 μl of cRGD) solution (5 mM, American Peptide Company, Sunnyvale, Calif.) was added. The reaction solution was stirred for 3-4 hours at 100 rpm. After conjugation, unreacted biomolecules were removed by three cycles of ultracentrifugation at 14,000 rpm and subsequent washing with PBS.

Attachment of siRNA to the FeCo/C NPs

For the siRNA to silence EGFP/EGFRvIII gene in U87-EGFP/EGFRvIII cells, the layer-by-layer (LbL) method was used. 1 ml of 1.0 mg/ml polyethyleneimine (PEI, MW 10,000) in distilled water was added to 0.5-1.0 mg carboxydextran FeCo/C NPs. After sonication for 2-4 hours under neutral pH, the coated particles were purified using ultracentrifugation and subsequent washing with distilled water. After characterization of the surface charge using zeta potential measurement (see FIG. 17), 10 μl of 0.1 mM siRNA solution in degassed PBS was added to 100 μl of PEI coated FeCo/C solution (5 mg/ml). After mild stirring for 1-2 hours, the NPs were purified by centrifugation and washing. Following this, another layer of PEI was deposited on the siRNA layer of FeCo/C-PEI with the same concentration and reaction volume as first PEI layer by stirring mildly for 1-2 hours. Sonication was avoided to protect the siRNA from heating and degradation. Final products was purified by ultracentrifugation (14,000 rpm) and washing with 1.0 mM NaCl solution. Surface charge was characterized using zeta potential measurement (Zetasizer Nano ZS, Malvern Instruments, Westborough, Mass.) to perform the siRNA transfection experiment. The zeta potential value increased from −20 mV to 40 my after the coating of a single layer of PEI on carboxydextran-coated FeCo/C NPs. Upon complexation of negatively charged siRNA molecules, the zeta potential value again dropped to −15 my. Further, on coating a second layer of PEI on the NPs, this value again became positive, thereby indicating a successful layer-by-layer coating of siRNA and PEI on the FeCo/C-dextran NPs.

### Cell Culture of U87-EGFP, U87-EGFRvIII and Other Control Cells

For the hyperthermia, MR imaging, Raman study and siRNA transfection experiments, EGFP or EGFRvIII over expressed U87 cells (U87-EGFP or U87-EGFRvIII) and several controls cells (PC-12 and Astrocytes) were cultured using previously reported methods, albeit with minor modifications. For U87-EGFP and U87-EGFRvIII cells, DMEM with high glucose, 10% FBS, 1% Streptomycin-penicillin and 1% Glutamax (Invitrogen, Carlsbad, Calif.) were used as basic components of growth media including Geneticin G418 (100 μg/ml, Invitrogen) as a selection marker for U87-EGFP and Hygromycin (100 μg/ml, Invitrogen) for U87-EGFRvIII. Astrocytes were cultured in the above mentioned growth media without any selection markers, while PC-12 cells were cultured in DMEM with 10% horse serum, 5% FBS and 1% Streptomycin-penicillin. All cells were cultured at 37° C. in humidified 5% CO₂ atmosphere.

### In Vitro Imaging

### MRI Imaging

For the MR imaging of the single layer of the cells with nanoparticles, U87-EGFP cells were grown on 15 mm diameter plastic cover slips (EMS, Hatfield, Pa.) in 12-well plates. After treatment with two different nanoparticles-FeCo/C MNPs and Fe₃O₄ MNPs (90-120 μg/ml) for 2 hours, cells were fixed with 4% paraformaldehyde solution. MRI imaging was performed at 4.7 T using the following parameters: point resolution: 156×156 μm, section thickness of 0.6 mm, TE=60 ms, TR=4000 ms, number of acquisitions=1.

### Raman Imaging

Internalization of nanoparticles into U87-EGFP cells was performed in 48-well plates using the above mentioned conditions. To each well. FeCo/C MNPs with different number of carbon graphite layers were added using the previously mentioned conditions. The cells were then imaged using a Renishaw Micro-Raman 2000 instrument with He—Ne laser excitation of 632.8 nm. The analysis of in vitro Raman imaging results, wherein confocal microscopy was used to observe the NP-internalized U87 cell lines, confirmed that the intensity of the D- and G-bands of the graphitic-carbon shells was indeed proportional to the thickness of the carbon shells on the FeCo/C NPs

### In Vive MR Imaging

11 nm FeCo/C NPs (10 μL, 0.25 mg of Fe) and Resovist (100 μL, 2.5 mg of Fe) were injected into a rat's tail vein. T₂-weighted MR images before injection and at 30 minute intervals post-injection were obtained using a 4.7 T MRI instrument (Bruker, Germany). The T₂-weighted MR signal intensity was also measured in the various organs over an extended period of 7 days. The following parameters were used: resolution of 234×256 μm, section thickness of 2.0 mm, TR=400 ms, TE=15 ms, number of acquisitions=8. Flip angle=30°. The nanoparticles were seen to localize in the liver, spleen and kidneys of the animal.

### Hyperthermia Measurements

All the AC magnetic field experiments were conducted using a Comdel CLF-5000 RF generator in a magnetic field with a frequency of 334 kHz and at amplitude of 150 Oe. To measure the temperature variation of the magnetic NPs suspensions, 2 mL of each suspension was taken in a double-walled test tube where the space between the outer and inner walls was evacuated to minimize any heat loss. The tube was placed at the center of the induction heater coil and an alcohol thermometer was used to measure the temperature increase in the suspension, thereby negating the electrical and magnetic effects of the generator on the thermometer. For hyperthermia measurements in cells, 15 mm diameter cover glass (Fisher scientific, Pittsburgh, Pa.) was autoclaved and sterilized under UV light. U87-EGFP or U87-EGFRvIII cells were cultured to 40-60% confluency on the cover glasses in 24-well plates. FeCo/C NPs or Fe₃(4 NPs in Opti-MEM (Invitrogen, Carlsbad, Calif.) were added at various concentrations (10, 30, 60, 90 μg/ml). After 2-4 hrs of incubation, cells were washed with DMEM to remove non-specific attachment of NPs and the media was changed to growth media. After overnight incubation, hyperthermia study was carried out. For target-specific hyperthermia experiments, control cells such as PC-12 and astrocytes were co-cultured with U87-EGFP (about 1:1 ratio). EGFR antibody or cRGD) conjugated FeCo/C NPs were used as a hyperthermia agents at different concentrations (1 μg/ml and 5 μg/ml). Following intracellular uptake of the aforementioned NP constructs, the cells were exposed to AC magnetic field for 15 min. Significant inhibition of proliferation and hyperthermia induced-cell death was observed mainly in the U87 cells while the less-tumorigenic PC-12 cells largely continued proliferating with time

siRNA Delivery and EGFP Knockdown Using FeCo/C NPs

PEI/siRNA/PEI-FeCo/C NPs were dispersed in the transfection media (Opti-MEM) at a final concentration of 30-120 μg/ml. 100 μl of the nanoparticle solution was added to the U87-EGFP cells at 50-60% confluency in 96-well plates. After 6-8 hours of incubation, the solution in each well was exchanged with the growth medium, followed by washing with DMEM. Fluorescence images were obtained at 48, 72 and 96 hours after transfection.

siRNA Delivery Against EGFRvIII and Combined Hyperthermia Treatment

U87-EGFRvIII cells were grown on 15 mm diameter cover glass in 24-well plates at 40-50/confluency. 30-120 μg/ml of PEI/siRNA/PEI-FeCo/C in the transfection media was added to the cells. After 2-4 hours of incubation, the wells were washed with DMEM and media replaced with growth medium. Hyperthermia treatment (334 kHz, 5 min) was performed at 72, 96 and 120 h post-siRNA treatment. MTS assay using the CellTiter 96 Aqueous One Solution (Promega, Madison, Wis.) was performed within 4 hours of incubation after hyperthermia (protocol as recommended by manufacturer) in order to quantify the synergistic cell death. Quantitative analysis based upon MTS assay showed that treatment of cells with siRNA-NPs against EGFRvIII followed by hyperthermia, induced significantly more cell death, as compared to the controls. This could be attributed to the fact that silencing of the EGFRvIII oncogene results in a decrease in expression of the focal adhesion proteins which makes the cells more susceptible to heat, thereby leading to a synergistic increase in cell death.

1. A nanoparticle comprising an iron cobalt core; a graphitic carbon shell surrounding the core; a biocompatible carbohydrate coating disposed on the shell; a cationic coating disposed on said biocompatible coating; and small interfering RNAs (siRNAs) disposed on said cationic coating, wherein said siRNAs are selected to interfere with expression of a gene in a tumor cell.

2. The nanoparticle of claim 1, wherein said carbohydrate of said biocompatible coating comprises functional groups for covalent attachment of tumor targeting agents; and said nanoparticle further comprises a targeting agent for said tumor cell conjugated to one of said functional groups.

3. The nanoparticle of claim 2, wherein said carbohydrate is dextran.

4. The nanoparticle of claim 2 wherein said targeting agent is an antibody, a peptide, a carbohydrate, a lipopolysaccharide, or a small molecule.

5. The nanoparticle of claim 4, wherein said antibody is a monoclonal antibody that binds to an epidermal growth factor receptor.

6. The nanoparticle of claim 4, wherein said targeting agent is a peptide containing a RGD sequence.

7. The nanoparticle of claim 1, wherein said cationic coating comprises polyethyleneimine.

8. A pharmaceutical composition comprising the nanoparticle of claim 1 and a pharmaceutically acceptable carrier.

9. A method to deliver a siRNA to the intracellular region of a cell of interest in a subject comprising administering a pharmaceutical composition comprising the nanoparticle of claim 2.

10. The method of claim 9, wherein the nanoparticle comprises a targeting agent for an epidermal growth factor receptor or a receptor that specifically binds to a peptide containing RGD.

11. The method of claim 9, wherein said cell is a glioblastoma cell.

12. A method to increase the temperature of a cell in a subject comprising administering a pharmaceutical composition comprising the nanoparticle of claim 2 and applying a magnetic field to the cells targeted by the targeting agent.

13. The method of claim 12, wherein said magnetic field is applied using a radiofrequency field generator.

14. A method to silence at least one gene in a subject comprising administering the pharmaceutical composition of claim 8.

15. The method of claim 14, wherein said gene is an epidermal growth factor receptor gene.

16. A method to detect a cell in a subject comprising administering a pharmaceutical composition comprising the nanoparticle of claim 2 and detecting said nanoparticle using an imaging device.

17. The method of claim 16 wherein said imaging device is a magnetic resonance imaging device or a Raman spectroscopy device.

18. A kit for detecting a cell in a biological sample or a subject comprising the nanoparticle of claim 1 and instructions for use.

19. A method of preparing a nanoparticle comprising an iron cobalt core and a graphitic carbon shell surrounding the core, said method comprising the steps of:
a. Dissolving iron and cobalt precursors with a carbohydrate in an aqueous solution;
b. Heating the solution under conditions sufficient for formation of nanoparticles; and
c. Annealing the nanoparticles under conditions suitable for the formation of a graphitic carbon shell on the nanoparticles.

20. The method of claim 19 further comprising the following steps:
d. Suspending the nanoparticles with a carbohydrate under basic conditions to provide a biocompatible coating on said shell;
e. Magnetically separating the coated nanoparticles from the solution;
f. Adding an aqueous solution of a cationic polymer to the biocompatible coated nanoparticles under conditions to provide a cationic coating on the biocompatible coated nanoparticles;
g. Purifying the nanoparticles;
h. Adding a solution of an siRNA to the nanoparticles;
i. Optionally repeating steps (f) and (g) to provide additional layers of the cationic coating.

21. The method of claim 19 wherein the aqueous solution is water.

22. The method of claim 19, wherein said conditions of step (b) comprise at a temperature between about 180-250° F. for about 7-10 hours.

23. The method of claim 19 wherein the conditions of step (c) comprise an argon atmosphere at a temperature between about 900-1100° F. for about 4-6 hours.
